(19) United States
(12) Patent Application Publication
(10) Pub. No.: US 2009/0239836 A1
(43) Pub. Date: Sep. 24, 2009

(54) Multifunctional Ophthalmic Compositions

(76) Inventors: Mary Lee Ciolkowski, Fairport, NY (US); Yan Huang, Webster, NY (US); Martin J. Coffey, Pittsford, NY (US)

Correspondence Address: Bausch & Lomb Incorporated, One Bausch & Lomb Place, Rochester, NY, 14604-2701, US

(21) Appl. No.: 12/398,202

(22) Filed: Mar. 5, 2009

Related U.S. Application Data
(60) Provisional application No. 61/038,821, filed on Mar. 24, 2008.

(51) Int. Cl. A61K 31/56; A61K 31/55; A61K 31/498; A61K 31/496; A61K 31/437; A61K 31/4709; A61K 47/10

(52) U.S. Cl. 514/179; 514/217.07; 514/249; 514/253.07; 514/300; 514/314; 514/772

(57) ABSTRACT

An multifunctional ophthalmic composition includes a nonionic oxygen-containing polymer and a surfactant. The composition can be used to treat or control ophthalmic diseases, conditions, or disorders. The composition can be a drug delivery vehicle for medicaments having low solubility in water.

### CROSS REFERENCE

This application claims the benefit of Provisional Patent Application No. 61/038,821 filed Mar. 24, 2008, which is incorporated by reference herein.

### BACKGROUND OF THE INVENTION

The present invention relates to multifunctional ophthalmic compositions. In particular, the present invention relates to ophthalmic compositions that are suitable for several different uses. More particularly, the present invention relates to such ophthalmic compositions for treatment or control of ophthalmic conditions or disorders.

Many ophthalmic compositions find therapeutic applications in the treatment or control of various ophthalmic conditions or disorders.

Treatment of diseases or disorders of the human eye is often accomplished through the topical administration of therapeutic agents. Any topical method of drug delivery must take into account and attempt to overcome many inherent physiological systems that operate to protect and maintain the vital front surfaces of the eye. In order to have therapeutic effect, a drug or other active ingredients must generally pass into the eye through the cornea. Problematic to topical delivery of drugs is the fact that the cornea is less permeable than the conjunctiva. Further, the surface area of the conjunctiva is highly vascular and is some fourteen times greater than the surface area of the cornea. For these reasons, the transconjunctival loss of instilled drugs is considerable. Further, water soluble drugs are quickly eliminated from the eye surface through tear outflow, a process that is often accelerated in the diseased eye, and topical delivery of drugs to the eye in sufficient quantity and for sufficient periods is often difficult. Thus, the effectiveness of prior art methods of topical drug application is often limited.

The most common known topical delivery of ophthalmic drugs is accomplished using water-based compositions, either as a solution or a suspension. Such compositions are generally delivered as drops or as a wash directly to the eye surface. However, the formulation of effective topical ophthalmic compositions faces many challenges. For example, such compositions should deliver an effective dose of an ophthalmic drug to the eye in view of the limited solubility of many drugs in aqueous media. The compositions should overcome the tendency to be rapidly cleared from the eye through the tear outflow. The compositions also should be comfortable to the patient and easy to apply, and avoid the inaccurate dosage problem due to drug segregation from the medium.

Various approaches have been applied in attempts to overcome these challenges. For example, suspensions of insoluble drugs have been prepared. However, such drugs tend to settle out of the medium and the compositions require vigorous resuspension by the patient immediately prior to application. Ointments have been used as vehicles for the delivery of water-insoluble ophthalmic drugs. However, ointments tend to be less comfortable and impair visual acuity due the excessively thick and uneven layer of material on the cornea. Moreover, ointments are difficult to apply since they often must be applied to the tarsal conjunctiva of the everted lower eye lid.

In addition to fluid-based vehicles, such as aqueous solutions, suspensions, and ointments, solid vehicles in the form of drug-releasing inserts have also been utilized to deliver drugs to the eye surface. Some inserts are hydrophilic contact lenses that have been impregnated with a drug that is released to the corneal surface over time after lens insertion. In other cases, the insert actually dissolves slowly to release the drug. The use of inserts, however, is not free from problems. They are cumbersome and carry the risk of eye infection by pathogens that may be introduced into the eye with the inserts.

Thus, although the challenges of formulating effective ophthalmic compositions have been met with some success, there is still a need for improved topical ophthalmic compositions.

### SUMMARY OF THE INVENTION

In general, the present invention provides improved multifunctional ophthalmic compositions.

In one aspect, the present invention provides topical compositions for the treatment of ophthalmic conditions, disorders, or diseases.

In another aspect, an ophthalmic composition of the present invention comprises a vehicle for an ophthalmic drug formulation.

In still another aspect, an ophthalmic composition of the present invention comprises a medicament that has low solubility in water, in an amount such that a therapeutically effective dose of the medicament can be delivered to the eye.

In yet another aspect, an ophthalmic composition of the present invention is capable of remaining on an ocular surface for an extended time.

In a further aspect, an ophthalmic composition of the present invention comprises a water-soluble nonionic oxygen-containing polymer, a surfactant, and water.

In yet another aspect, the nonionic oxygen-containing polymer is selected from the group consisting of polyethylene glycol, polypropylene glycol, polyoxyethylene-polyoxypropylene block copolymer, and mixtures thereof.

In still another aspect, the composition further comprises a tonicity-adjusting agent.

In still another aspect, the medicament is present in the composition in an amount in the range from about 0.01 mg/g to about 200 mg/g.

In yet another aspect, the medicament is selected from group consisting of anti-inflammatory agents, anti-infective agents (including antibacterial, antifungal, antiviral, antiprotozoal agents), anti-allergic agents, antiproliferative agents, anti-angiogenic agents, antiglaucoma agents, anti-oxidants, antihypertensive agents, neuroprotective agents, cell receptor agonists, cell receptor antagonists, immunomodulating agents, immunosuppressive agents, intraocular (“IOP”) lowering agents, beta adrenoceptor antagonists, alpha-2 adrenoceptor agonists, carbonic anhydrase inhibitors, cholinergic agonists, prostaglandins and prostaglandin receptor agonists, angiotensin converting enzyme (“ACE”) inhibitors, AMPA receptor antagonists, NMDA antagonists, angiotensin receptor antagonists, antihistamines, mast cell stabilizers or degranulation inhibitors, alpha-adrenergic receptor blockers, alpha-2 adrenoceptor antagonists, thromboxane A2 mimetics, protein kinase inhibitors, prostaglandin F derivatives, prostaglandin-2 alpha antagonists, cyclooxygenase-2 inhibitors, muscarinic agonists, and combinations thereof.

In a further aspect, the pharmaceutical composition has a viscosity of between about 2 centipoises (“cp” or mPa·s) to about 10,000 cp.

In another aspect of the present invention, a method of preparing an ophthalmic pharmaceutical composition comprises combining a medicament with a nonionic oxygen-containing polymer and a surfactant.

In still another aspect, the method further comprising reducing a size of the medicament while mixing with said nonionic oxygen-containing polymer and said surfactant.

In a further aspect, the present invention provides a method of treating or controlling an ocular disease, disorder, or condition. The method comprises administering a therapeutically amount of a composition that comprises a nonionic oxygen-containing polymer and a surfactant to an ocular tissue in need of such treatment or control.

In yet another aspect, the composition employed in such a method further comprises an ophthalmic medicament.

These and other features and advantages of the present invention will be further understood and appreciated by those skilled in the art by reference to the following detailed description and claims.

### DETAILED DESCRIPTION OF THE INVENTION

As used herein, the term “control” also includes reduction, amelioration, alleviation, and prevention.

As used herein, the phrase “low solubility in water” or “low aqueous solubility” means solubility in water of less than 0.01 mg/g at physiological pH (about 7.4) and at about 25° C. Although compositions and methods of the present invention are particularly applicable to medicaments or compounds having such solubility, such compositions and methods are also useful in providing novel formulations of enhanced concentrations of pharmaceutical compounds, which have solubility in water in the range of less than I mg/g and are difficult to be formulated into compositions having therapeutically significant concentrations.

In still other embodiments, compositions and methods of the present invention are also useful for medicaments or compounds having solubility in water (at pH of about 7.4 and temperature of about 25° C.) greater than about 1 mg/g, for example, when such compositions provide some desirable properties.

Throughout this disclosure, unless otherwise specified, concentrations of an ingredient of the composition or formulation are in weight percent.

In general, the present invention provides improved multifunctional ophthalmic compositions.

In one aspect, the present invention provides topical compositions for the treatment of ophthalmic conditions, disorders, or diseases.

In another aspect, an ophthalmic composition of the present invention comprises a vehicle for an ophthalmic drug formulation.

In still another aspect, an ophthalmic composition of the present invention may be administered to an eye of a patient who suffers discomfort or irritation of the eye; for example, as a result of a dry eye condition. In such a case, the ophthalmic composition may not include an ophthalmic drug.

In yet another aspect, an ophthalmic composition of the present invention is capable of remaining on an ocular surface for an extended time.

In a further aspect, an ophthalmic composition of the present invention comprises a water-soluble nonionic oxygen-containing polymer, a surfactant, and water.

hi still another aspect, an ophthalmic composition of the present invention further comprises a medicament that has low solubility in water, in an amount such that a therapeutically effective dose of the medicament can be delivered to the eye.

In still another aspect, the medicament is present in the composition in an amount in the range from about 0.01 mg/g to about 200 mg/g. Alternatively, the medicament is present in the composition in an amount in the range from about 0.01 mg/g to about 100 mg/g, or from about 0.01 mg/g to about 50 mg/g, or from about 0.01 mg/g to about 20 mg/g, or from about 0.01 mg/g to about 10 mg/g, from about 0.01 mg/g to about 1 mg/g, or from about 0.1 mg/g to about 100 mg/g, or from about 0.1 mg/g to about 50 mg/g, or from about 0.1 mg/g to about 20 mg/g, or from about 0.1 mg/g to about 10 mg/g, or from about 0.5 mg/g to about 50 mg/g, or from about 0.5 mg/g to about 20 mg/g, or from about 0.5 mg/g to about 10 mg/g, or from about 0.5 mg/g to about 5 mg/g.

In yet another aspect, the nonionic oxygen-containing polymer is selected from the group consisting of polyethylene glycol, polypropylene glycol, polyoxyethylene-polyoxypropylene block copolymer, and mixtures thereof.

In still another aspect, the nonionic oxygen-containing polymer is selected from the group consisting of polyethylene glycols having a molecular weight in the range from about 300 to about 20000. Alternatively, the nonionic oxygen-containing polymer is selected from the group consisting of polyethylene glycols having a molecular weight in the range from about 600 to about 10000, or from about 1000 to about 8000. Non-limiting examples of such polyethylene glycol are known under the common names of PEG-400, PEG-600, PEG-1000, PEG-2000, PEG-3350, PEG-4000, PEG-6000, PEG-8000, PEG-10000, and PEG-20000. Suitable polyethylene glycols having molecular weight in this range are known under the CTFA (Cosmetic, Toiletry and Fragrance Association) nomenclature as PEG-8, PEG-12, PEG-20, PEG-32, PEG-75, PEG-100, and PEG-150 with molecular weight of 400, 600, 1000, 1450, 3350, 4500, and 8000, respectively. Particularly suitable polyethylene glycols are those having molecular weight in the range from about 2000 to about 8000.

In yet another aspect, the nonionic oxygen-containing polymer is selected from the group consisting of polypropylene glycols having a molecular weight in the range from about 300 to about 10000. Alternatively, the nonionic oxygen-containing polymer is selected from the group consisting of polypropylene glycols having a molecular weight in the range from about 400 to about 8000, or from about 1000 to about 4000. Non-limiting examples of such polyethylene glycol are known under the CTFA nomenclature of PPG-9, PPG-10, PPG-17, PPG-20, PPG-26, PPG-55, and PPG30 having molecular weight of 425, 700, 1000, 1200, 2000, 3000, and 4000, respectively.

In a further aspect, the nonionic oxygen-containing polymer is selected from the group consisting of polyoxyethylene-polyoxypropylene block copolymers. Several of these copolymers are known under the name of Poloxamer. Non-limiting examples of such block copolymers include Pluronic® L44NF, F68NF, F87NF, F108NF, and F127NF.

The amount of a nonionic oxygen-containing polymer in a composition of the present invention is in the range from about 0.1 to about 25 percent by weight. Alternatively, the amount of a nonionic oxygen-containing polymer in a composition of the present invention is in the range from about 0.5 to about 15 percent, or from about 0.5 to about 12 percent, or from about 0.5 to about 10 percent, or from about 0.5 to about 8 percent, or from about 0.5 to about 5 percent, from about 0.5 to about 3 percent, or from about 3 to about 25 percent, or from about 3 to about 15 percent, or from about 5 to about 25 percent, or from about 5 to about 15 percent, by weight. In one aspect, the amount of the polymer included in a composition varies in inverse relationship with its molecular weight.

In yet another aspect, the surfactant included in a composition of the present invention comprises a nonionic surfactant, an anionic surfactant, a cationic surfactant, a phospholipid, or a combination thereof.

Non-limiting examples of nonionic surfactants include polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamines (synthetic block polymers of ethylene oxide and propylene oxide attached to ethylene diamine, such as those commonly known by their trade names of Tetronic(®; e.g., Tetronic® 1508 or Tetronic® 908, etc., other nonionic surfactants such as Brij®, Myrj®, and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). Such compounds are delineated in Martindale, 34^{th} ed., pp. 1411-1416 (Martindale, “The Complete Drug Reference,” S. C. Sweetman (Ed.), Pharmaceutical Press, London, 2005) and in Remington, “The Science and Practice of Pharmacy,” 21^{st} Ed., p. 291 and the contents of chapter 22, Lippincott Williams & Wilkins, New York, 2006).

Suitable anionic surfactants are those containing carboxylate, sulfonate, and sulfate ions. The chain length of fatty acids ranges from 12 to 8 carbon atoms. Long alkyl chain sulfonates (12-18 carbon atoms), as well as alkyl aryl sulfonates such as sodium dodecylbenzene sulfonate, may be used. The sulfonate ion is less subject to hydrolysis and precipitation in the presence of multivalent ions. A suitable group of sulfonates are the dialkyl sodium sulfosuccinates, particularly sodium bis-(2-ethylhexyl)sufosuccinate. Suitable sulfate surfactants include sodium lauryl sulfate.

Suitable cationic surfactants include long-chain (12-18 carbon atoms) cations, such as amine salts and quaternary ammonium salts, such as the alkyl benzyldimethylammonium chlorides (alkyl chain having 8-16 carbon atoms).

Non-limiting examples of phospholipids include lecithins containing the L-α-glycerophosphoylcholine esterified to two long-chain fatty acids (often oleic, palmitic, stearic, and linoleic), dipalmitoylphosphatidylcholine (“DPPC”), and phosphatidylglycerol.

The concentration of a surfactant in a composition of the present invention can be in the range from about 0.001 to about 5 percent by weight (or alternatively, from about 0.01 to about 5, or from about 0.01 to about 2, or from about 0.01 to about 1, or from about 0.01 to about 0.5, or from about 0.1 to about 5, or from about 0.1 to about 2, or from about 0.001 to about 0.1, or from about 0.001 to about 0.05, or from about 0.5 to about 5, or from about 0.5 to about 2, or from about 1 to about 5, or from about 1 to about 3 percent by weight).

In still another aspect, the composition further comprises a tonicity-adjusting agent. Non-limiting examples of such tonicity-adjusting agents include sodium and potassium chloride, glycerin, dextrose, mannose, mannitol, sorbitol, and calcium and magnesium chloride. These agents are typically used individually in amounts ranging from about 0.01 to about 3 percent by weight; preferably, from about 0.1 to about 2 percent by weight. Preferably, the tonicity agent is employed in an amount to provide a final osmolality of from about 200 to about 400 mOsm/kg; preferably, between about 200 and about 350 mOsm/kg; more preferably, between about 240 to about 320 mOsm/kg.

A variety of medicaments known within the pharmaceutical industry are suitable for use in accordance with the teachings of the present invention. Preferred medicaments are those utilized in treating ocular indications, diseases, syndromes, injuries, and the like. In addition, although not wanting to be bound by any particular theory, Applicant believes that the present invention is particularly suited for use with medicaments that are water insoluble or poorly water-soluble, but are solubilizable in water-miscible materials. Thus, the present invention provides enhancements to the delivery, bioavailability and target tissue concentrations of such insoluble or poorly soluble medicaments.

Non-limiting examples of medicaments, including water-insoluble or poorly water soluble medicaments, especially those for use in an ocular environment according to the teachings of the present invention, include, but are not limited to, anti-inflammatory agents, anti-infective agents (including antibacterial, antifungal, antiviral, antiprotozoal agents), anti-allergic agents, antiproliferative agents, anti-angiogenic agents, anti-oxidants, antihypertensive agents, neuroprotective agents, cell receptor agonists, cell receptor antagonists, immunomodulating agents, immunosuppressive agents, IOP lowering agents, beta adrenoceptor antagonists, alpha-2 adrenoceptor agonists, carbonic anhydrase inhibitors, cholinergic agonists, prostaglandins and prostaglandin receptor agonists, angiotensin converting enzyme (“ACE”) inhibitors, AMPA receptor antagonists, NMDA antagonists, angiotensin receptor antagonists, somatostatin agonists, mast cell degranulation inhibitors, alpha-adrenergic receptor blockers, alpha-2 adrenoceptor antagonists, thromboxane A2 mimetics, protein kinase inhibitors, prostaglandin F derivatives, prostaglandin-2 alpha antagonists, cyclooxygenase-2 inhibitors, muscarinic agents, and combinations thereof.

In one embodiment, the medicament is selected from the group consisting of anti-inflammatory agents, anti-infective agents (including antibacterial, antifungal, antiviral, antiprotozoal agents), anti-allergic agents, antiproliferative agents, anti-angiogenic agents, antiglaucoma agents, anti-oxidants, antihypertensive agents, neuroprotective agents, cell receptor agonists, cell receptor antagonists, immunomodulating agents, immunosuppressive agents, IOP lowering agents, and combinations thereof.

In another embodiment, the medicament is selected from the group consisting of anti-inflammatory agents, antiproliferative agents, anti-angiogenic agents, neuroprotective agents, immunomodulating agents, IOP lowering agents, and combinations thereof.

In still another embodiment, the medicament is selected from the group consisting of beta adrenoceptor antagonists, alpha-2 adrenoceptor agonists, carbonic anhydrase inhibitors, cholinergic agonists, and prostaglandin receptor agonists.

In a further embodiment, the medicament is selected from the group consisting of prostaglandin agonist, beta-2 agonist, muscarinic antagonist, and combinations thereof.

In one embodiment, the medicament comprises a fluoroquinolone having Formula I (a new-generation fluoroquinolone antibacterial agent, disclosed in U.S. Pat. No. 5,447,926, which is incorporated herein by reference).

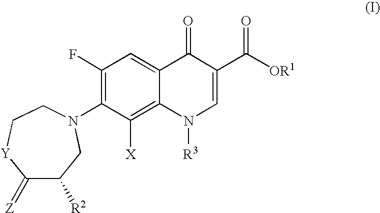

wherein R¹ is selected from the group consisting of hydrogen, unsubstituted lower alkyl groups, substituted lower alkyl groups, cycloalkyl groups, unsubstituted C₅-C₂₄ aryl groups, substituted C₅-C₂₄ aryl groups, unsubstituted C₅-C₂₄ heteroaryl groups, substituted C₅-C₂₄ heteroaryl groups, and groups that can be hydrolyzed in living bodies; R² is selected from the group consisting of hydrogen, unsubstituted amino group, and amino groups substituted with one or two lower alkyl groups; R³ is selected from the group consisting of hydrogen, unsubstituted lower alkyl groups, substituted lower alkyl groups, cycloalkyl groups, unsubstituted lower alkoxy groups, substituted lower alkoxy groups, unsubstituted C₅-C₂₄ aryl groups, substituted C₅-C₂₄ aryl groups, unsubstituted C₅-C₂₄ heteroaryl groups, substituted C₅-C₂₄ heteroaryl groups, unsubstituted C₅-C₂₄ aryloxy groups, substituted C₅-C₂₄ aryloxy groups, unsubstituted C₅-C₂₄ heteroaryloxy groups, substituted C₅-C₂₄ heteroaryloxy groups, and groups that can be hydrolyzed in living bodies; X is selected from the group consisting of halogen atoms; Y is selected from the group consisting of CH₂, O, S, SO, SO₂, and NR⁴, wherein R⁴ is selected from the group consisting of hydrogen, unsubstituted lower alkyl groups, substituted lower alkyl groups, and cycloalkyl groups; and Z is selected from the group consisting of oxygen and two hydrogen atoms.

In another embodiment, the medicament comprises a fluoroquinolone having Formula II.

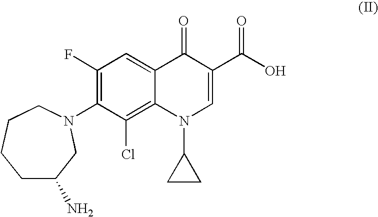

((R)-(+)-7-(3-amino-2,3,4,5,6,7-hexahydro-1H-azepin-1-yl)-8-chloro-1-cyclopropyl-6-fluoro-1,4-dihydro4-oxoquinoline-3-carboxylic acid).

In yet another aspect, the medicament comprises a quinolone or an analog thereof, such as cinoxacin, ciprofloxacin, clinafloxacin, difloxacin, enoxacin, fleroxacin, flumequine, gatifloxacin, grepafloxacin, levofloxacin, lomefloxacin, miloxacin, moxifloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, or trovafloxacin.

In still another embodiment, the medicament comprises a glucocorticoid receptor agonist having Formulae III or IV, as disclosed in US Patent Application Publication 2006/0116396, which is incorporated herein by reference.

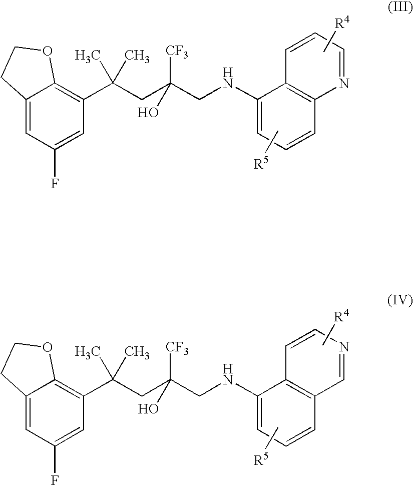

wherein R⁴ and R⁵ are independently selected from the group consisting of hydrogen, halogen, cyano, hydroxy, C₁-C₁₀ (alternatively, C₁-C₅ or C₁-C₃) alkoxy groups, unsubstituted C₁-C₁₀ (alternatively, C₁-C₅ or C₁-C₃) linear or branched alkyl groups, substituted C₁-C₁₀ (alternatively, C₁-C₅ or C₁-C₃) linear or branched alkyl groups, unsubstituted C₃-C₁₀ (alternatively, C₃-C₆ or C₃-C₅) cyclic alkyl groups, and substituted C₃-C₁₀ (alternatively, C₃-C₆ or C₃-C₅) cyclic alkyl groups.

In yet another embodiment, the medicament comprises a glucocorticoid receptor agonist having Formula V (a species of compound having Formula III).

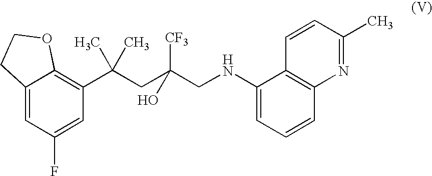

Other compounds that can function as glucocorticoid agonists and methods for their manufacture are disclosed, for example, in U.S. Patent Application Publications 2004/0029932, 2004/0162321, 2004/0224992, 2005/0059714, 2005/0176706, 2005/0203128, 2005/0234091, 2005/0282881, 2006/0014787, 2006/0030561, 2006/0116396, 2006/0189646, and 2006/0189647, all of which are incorporated herein by reference in their entirety.

In some embodiments, the medicament comprises other anti-inflammatory agents, such as soft steroids (e.g., loteprednol etabonate) or non-steroidal anti-inflammatory drugs. Non-limiting examples of the NSAIDs are: aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), ε-acetamidocaproic acid, S-(5′-adenosyl)-L-methionine, 3-amino-4-hydroxybutyric acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, their physiologically acceptable salts, combinations thereof, and mixtures thereof.

A viscosity-adjusting agent can be included in a composition of the present invention to facilitate the administration of the composition into the subject or to promote the bioavailability in the subject for the intended time period of treatment. A viscosity-adjusting agent can be a low or high molecular weight material. In one aspect, the viscosity of the composition or formulation is in the range from about 5 cp (centipoise or mPa·s) to about 5000 cp. Alternative the viscosity of the composition or formulation is in the range from about 10 cp to about 5000 cp, or from about 10 cp to about 2000 cp, or from about 10 cp to about 1000 cp, or from about 10 cp to about 500 cp. Non-limiting examples of viscosity-adjusting agents include derivatives of cellulose, such as carboxymethyl cellulose, hydroxypropylmethyl cellulose, hydroxypropyl cellulose, and poly(acrylic acid)-based polymers, such as Carbopol® (poly(acrylic acid) crosslinked with allyl sucrose; e.g., 71G NF, 971P NF, 974P NF, 980 NF, 981 NF, or 941 NF), Permulen® (poly(acrylic acid) modified by long chain (C₁₀-C₃₀) alkylacrylates and crosslinked with allylpentaerythritol; e.g., TR-1 NF or TR-2 NF), polycarbophil (poly(acrylic acid) crosslinked with divinyl glycol), and Carbomer (poly(acrylic acid) crosslinked with polyalkenyl polyether) polymers. Other viscosity-adjusting agents include medium-chain triglycerides (“MCT”, wherein the fatty acyl moiety comprises 4-12 carbon atoms), long-chain triglycerides (“LCT,” wherein the fatty acyl moiety has more than 12, preferably more than 18, and more preferably more than 22, carbon atoms, but not long enough to segregate into a different phase), polysaccharides, such as alginate and its salts, hyaluronic acid and its salts, chondroitin sulfate and its salts, dextrans, such as, dextran 70, water soluble proteins, such as gelatin, vinyl polymers, such as, polyvinyl alcohol, polyvinylpyrrolidone, povidone, and polysiloxanes.

In one or more embodiments of the present invention, the composition can also include one or more additives, including, but not limited to, preservatives, non-anti-oxidants, chelating agents, solubility-enhancing agents, buffers, and combinations thereof.

Non-limiting examples of preservatives include benzalkonium chloride (BAK”), quaternary ammonium compounds (e.g., polyquat-1, polyquat-10), hydrogen peroxide, urea hydrogen peroxide, sorbic acid/EDTA (ethylenediamine tetraacetic acid), p-hydroxybenzoic acid esters, polyhexamethylene biguanide (“PHMB”), phenylethyl alcohol, ethylparaben, and methylparaben. These agents may be present in individual amounts of from about 0.001 to about 2 percent by weight (preferably, about 0.001 to about 0.5 percent by weight).

A non-limiting example of solubility-enhancing agents is beta-cyclodextrin.

Physiologically acceptable buffers include, but are not limited to, a phosphate buffer or a Tris-HCl buffer (comprising tris(hydroxymethyl)aminomethane and HCl). For example, a Tris-HCl buffer having pH of 7.4 comprises 3 g/l of tris(hydroxymethyl)aminomethane and 0.76 g/l of HCl. In yet another aspect, the buffer is 10× phosphate buffer saline (“PBS”) or 5× PBS solution.

Other buffers also may be found suitable or desirable in some circumstances, such as buffers based on HEPES (N-{2-hydroxyethyl}peperazine-N′-{2-ethanesulfonic acid}) having pKₐ of 7.5 at 25 ° C. and pH in the range of about 6.8-8.2; BES (N,N-bis{2-hydroxyethyl}2-aminoethanesulfonic acid) having pKₐ of 7.1 at 25° C. and pH in the range of about 6.4-7.8; MOPS (3-{N-morpholino}propanesulfonic acid) having pKₐ of 7.2 at 25° C. and pH in the range of about 6.5-7.9; TES (N-tris{hydroxymethyl}-methyl-2-aminoethanesulfonic acid) having pKₐ of 7.4 at 25° C. and pH in the range of about 6.8-8.2; MOBS (4-{N-morpholino}butanesulfonic acid) having pKₐ of 7.6 at 25° C. and pH in the range of about 6.9-8.3; DIPSO (3-(N,N-bis{2-hydroxyethyl}amino)-2-hydroxypropane)) having pKₐ of 7.52 at 25° C. and pH in the range of about 7-8.2; TAPSO (2-hydroxy-3{tris(hydroxymethyl)methylamino}-1-propanesulfonic acid)) having pKₐ of 7.61 at 25° C. and pH in the range of about 7-8.2; TAPS ({(2-hydroxy-1,1-bis(hydroxymethyl)ethyl)amino}-1-propanesulfonic acid)) having pKₐ of 8.4 at 25° C. and pH in the range of about 7.7-9.1; TABS (N-tris(hydroxymethyl)methyl-4-aminobutanesulfonic acid) having pKₐ of 8.9 at 25° C. and pH in the range of about 8.2-9.6; AMPSO (N-(1,1-dimethyl-2-hydroxyethyl)-3-amino-2-hydroxypropanesulfonic acid)) having pKₐ of 9.0 at 25° C. and pH in the range of about 8.3-9.7; CHES (2-cyclohexylamino)ethanesulfonic acid) having pKₐ of 9.5 at 25° C. and pH in the range of about 8.6-10.0; CAPSO (3-(cyclohexylamino)-2-hydroxy-1-propanesulfonic acid) having pKₐ of 9.6 at 25° C. and pH in the range of about 8.9-10.3; or CAPS (3-(cyclohexylamino)-1-propane sulfonic acid) having pKₐ of 10.4 at 25° C. and pH in the range of about 9.7-11.1.

In certain embodiments, a composition of the present invention is formulated in a buffer having pH in the range from about 4 to about 8. In other embodiments, such pH is in the range from about 4 to about 6.8, or alternatively, from about 5 to about 6.8. In such embodiments, the buffer capacity of the composition desirably allows the composition to come rapidly to a physiological pH after being administered into the patient. In still other embodiments, the pH of the composition is in the range from about 7 to 7.5.

In one aspect, the pharmaceutical composition can remain in the ocular environment for an extended time and releases the medicament over a period of time. In one embodiment, the pharmaceutical composition can release the medicament over a period of 4 hours, 8 hours, or longer. In another embodiment, the pharmaceutical composition can release the medicament over a period of 12 hours or longer. In another preferred embodiment, the pharmaceutical composition can release the medicament over a period of 24 hours or longer. In another embodiment, the pharmaceutical composition can release the medicament over a period of 2, 3, 4, 5, 6, or 7 days or longer. In another preferred embodiment, the pharmaceutical composition can release the medicament over a period of 2, or 4 weeks or longer.

In one aspect, a composition of the present invention is formulated for topical administration. In one embodiment, such a composition is formulated for topical administration to the anterior segment of the eye, such as to the anterior ocular surface or the conjunctiva, for treating or controlling an anterior-segment disease, disorder, or condition.

In one aspect, a pharmaceutical composition of the present invention is suitable for treating ocular diseases, disorders, or conditions via ocular injection (e.g., intravitreal injection).

Alternatively, the composition can be formulated for injection into an ocular environment, including, but not limited to, the vitreous cavity or the subconjunctiva of an eye within a human or an animal. The composition can be formulated for ocular injection according to known methods and principles, and then injected using an injection delivery device such as an appropriately gauged needle; for example, 25-30 gauge needle.

Before the mixture is injected into an ocular environment, the composition is desirably sterilized. Suitable methods of sterilization include, but are not limited to, sterile filtration, thermal sterilization, and gamma irradiation. Where sterile filtration is selected, one suitable method of sterile filtration can utilize a filter having a pore size of at least about 0.2 micrometer or less. Where thermal sterilization is selected, one suitable method of thermal sterilization can include sterilizing the mixture at a temperature of at least about 150 ° C. for a period of at least about 25 minutes. Where gamma irradiation is selected, one suitable method can include exposure of the compositions of the present invention to gamma rays at a level of from about 2.5 Mrad to about 3.5 Mrad.

As noted above, another aspect of the present invention involves a method of treating an ocular disease, disorder, or condition. The method includes administering a pharmaceutical composition comprising a water-soluble nonionic oxygen-containing polymer and a surfactant into an ocular environment. The nonionic oxygen-containing polymer and the surfactant are chosen among those disclosed hereinabove.

In one aspect, the composition used to carry out the method further includes an ophthalmic medicament. The medicament can be chosen to treat the specific disease, condition, or disorder of the situation.

The composition can be used to treat an ocular disease, disorder, or condition including, but not limited to diabetic retinopathy, diabetic macular edema, cystoid macular edema, age macular degeneration (including the wet and dry form), optic neuritis, retinitis, chorioretinitis, intermediate and posterior uveitis, choroidal neovascuralization, and combinations thereof.

In another aspect, a composition of the present invention including an appropriate medicament is used to treat or control a ocular disease, condition, or disorder of the anterior segment, including dry eye (also known as kerotoconjunctivitis sicca), anterior uveitis (including iritis and iridocyclitis), keratitis, conjunctivitis, keratoconjunctivitis (including vernal keratoconjunctivitis (or “VKC”) and atopic keratoconjunctivitis), corneal ulcer, corneal edema, sterile corneal infiltrates, anterior scleritis, episcleritis, blepharitis, and post-operative (or post-surgical) ocular inflammation resulting from procedures such as photorefractive keratectomy, cataract removal surgery, intraocular lens (“IOL”) implantation, laser-assisted in situ keratomileusis (“LASIK”), conductive keratoplasty, and radial keratotomy.

The nonionic oxygen-containing polymer, surfactant, medicament, and other optional ingredients can be combined to form any suitable mixture, including, but not limited to, a solution, a semi-solid, or a suspension. In another embodiment, the solution can further be added to a hydrophobic medium and the total can be formed into a stable emulsion. For example, the mixture can be a suspension containing particles of the medicament in the medium containing the nonionic oxygen-containing polymer and surfactant. In various embodiments of the present invention, the particles of the medicament have a particle size of between about 0.01 μm to about 4 μm in diameter. In another embodiment, the predominant particle size is between about 0.05 μm to about 2 μm in diameter. In still another embodiment, the median particle size is between about 1 μm to about 2 μm in diameter. In yet another embodiment, the median particle size is about 1.5-1.7 μm.

A composition of the present invention as a drug delivery vehicle of the present invention can address one or more of the challenges described herein regarding the delivery of therapeutically meaningful amounts of medicaments to target tissues within the ocular environment.

For example, solubilization of a medicament that typically has a low solubility in an aqueous medium can have a higher solubility in a composition of the present invention. Such increased solubility can enhance the availability of that medicament or medicament particles at, in, or near those target tissues, and thereby enhance the medicament's concentration at, in, or near the target tissues.

For example, the solubility of the compound having Formula V in some embodiments of the present composition is compared to that in water in Table 1.

TABLE 1
Solubility of Compound Having Formula V in Various Media

| Medium | Solubility (μg/mL) |
|---|---|
| Water, pH 7 | 0.2 |
| 1% Polysorbate 80 | 12 |
| PEG-400 | 5.6 |
| 10% PEG-400/1% polysorbate 80 (a | 12 |
| composition of the present invention) | |
| 25% PEG-40/1% polysorbate 80 (a | 18 |
| composition of the present invention) | |

In some instances, the amount or dose of the medicament can be completely soluble in the present medium such that the entire amount or dose is delivered as a solution to the desired ocular environment. In other instances, the medicament can be delivered as a suspension, yet because of the higher solubility in the delivery vehicle of the present invention, the concentration of the medicament in the fluid phase of the composition can be high and thus, a more significant concentration of the medicament is available at or near the target tissue.

An additional advantage of using a composition of the present invention is the improved potential for the bioavailability of particles. As the medium of the present composition dissipates, or as the ocular fluid (such as tear or vitreous humor) penetrates the composition droplet or injection bolus, very small particles of the medicament are exposed. Under most conditions, smaller particles of a medicament have higher bioavailability than larger particles. An added advantage of smaller particles is that they are less likely to migrate into the visual axis and occlude vision unlike conventional ocular compositions such as an ointment or ocular injectable dispersion.

The following examples further illustrate the present invention and are not to be construed as limiting the invention or scope of the specific procedures or compositions described herein.

TABLE 2
Suspensions Comprising Glucocorticoid Receptor Agonist

| | Example | | | | | | | |
|---|---|---|---|---|---|---|---|---|
| | 1 | | 2 | | 3 | | 4 | |
| Lot Number | 2604-MJC- | | 2604-MJC- | | 2604-MJC- | | 2604-MJC- | |
| | 077-60 | | 077-30 | | 077-10 | | 074-V | |
| Compound having | 60 | | 30 | | 10 | | 0 | |
| Formula V | | | | | | | | |
| PEG-3350 NF | 94 | | 97 | | 99 | | 100 | |
| Polysorbate 80 NF | 9.4 | | 9.7 | | 9.9 | | 10 | |
| Boric acid NF | 5.076 | | 5.238 | | 5.346 | | 5.4 | |
| Sodium phosphate | 1.711 | | 1.765 | | 1.802 | | 1.82 | |
| dibasic | | | | | | | | |
| Sodium phosphate | 0.818 | | 0.844 | | 0.861 | | 0.87 | |
| monobasic | | | | | | | | |
| Polyhexamethylene | 0.001 | | 0.001 | | 0.001 | | 0.001 | |
| biguanide | | | | | | | | |
| (“PHMB”) | | | | | | | | |
| Butylated hydroxy | 0.094 | | 0.097 | | 0.099 | | 0.1 | |
| toluene (“BHT”, an | | | | | | | | |
| antioxidant) | | | | | | | | |
| EDTA | 0.094 | | 0.097 | | 0.099 | | 0.1 | |
| Water | q.s. to 1000 | g | q.s. to 1000 | g | q.s. to 1000 | g | q.s. to 1000 | g |

Note:
all quantities in Table 2 are in grams.

The suspension of Example 1 was prepared by the process comprising the following steps:

Preparing sterile suspension vehicle by dissolving all the ingredients except the compound having Formula V (the drug substance) in about 140 g of water with stirring and then sterile filtering through a 0.2 μm polyethersulfone filter membrane;

Adding sterile vehicle to sterile milling vessel containing drug substance and sterile polystyrene beads; wet-milling drug in suspension vehicle by mixing in AR-500 milling apparatus at 1000 rpm for 3-30 minute cycles; (drug concentration in the vehicle during the bead-milling was about 24 percent by weight); and

Quantitatively transferring, in the biological safety cabinet, concentrated suspension and the PS beads used for wet-milling to a filtration apparatus for separating the suspension from the beads and diluting with additional water to the highest concentration (60 mg/g).

The suspension of Examples 2 and 3 were obtained by performing dilution of the suspension of Example 1 to create lower suspension concentrations (10, 30 mg/g).

A summary of some of the analytical results for the formulations of Table 2 is shown in Table 3.

TABLE 3
Some Analytical Results of Suspensions of Examples 1-4

| | Lot Number | | | |
|---|---|---|---|---|
| | 2604-MJC- | 2604-MJC- | 2604-MJC- | 2604-MJC- |
| | 077-60 | 077-30 | 077-10 | 074-V |
| pH | 7.1 | 7.1 | 7.1 | 7.1 |
| Osmolality | 291 | 291 | 283 | 284 |
| (mOsm/kg) | | | | |
| Assay of drug | 101.9% | 101.8% | 101.3% | 0 |
| substance | | | | |
| Median particle | 1.6 | 1.6 | 1.5 | Not |
| diameter (μm) | | | | applicable |
| Particle size by | <0.6 (10 vol | <0.6 (10 vol | <0.6 (10 vol | Not |
| light scattering | %) | %) | %) | applicable |
| (μm) | <1.0 (25 vol. | <0.9 (25 vol. | <0.8 (25 vol. | |
| | %) | %) | %) | |
| | <1.5 (50 vol. | <1.4 (50 vol. | <1.4 (50 vol. | |
| | %) | %) | %) | |
| | <2.1 (75 vol. | <2.1 (75 vol. | <1.9 (75 vol. | |
| | %) | %) | %) | |
| | <2.8 (90 vol. | <2.8 (90 vol. | <2.6 (90 vol. | |
| | %) | %) | %) | |
| | <3.3 (95 vol. | <3.4 (95 vol. | <3.1 (95 vol. | |
| | %) | %) | %) | |

Other suspensions may be made in a similar manner.

### EXAMPLE 5

Other Suspensions Comprising Compound Having Formula V

| Ingredient | Weight (g) | |
|---|---|---|
| PEG 3350 | 10 | |
| Polysorbate 80 | 1 | |
| BHT antioxidant | 0.01 | |
| Potassium chloride | 0.04 | |
| Sodium chloride | 0.2 | |
| EDTA dihydrate | 0.01 | |
| Benzalkonium chloride (preservative) | 0.02 | |
| Na₂HPO₄ (anhydrous) | 0.182 | |
| NaH₂PO₄ (anhydrous) | 0.087 | |
| Compound having Formula V | 0.001-1 | |
| Water | q.s. to 100 | g |

Still other examples of compositions of the present invention are shown below.

### EXAMPLES 6-7

Other Drug Delivery Vehicles

| | Example | | | |
|---|---|---|---|---|
| | 6 | | 7 | |
| Boric acid NF | | | | |
| Na₂HPO₄ (anhydrous) | 0.182 | | 0.182 | |
| NaH₂PO₄ (anhydrous) | 0.1 | | 0.1 | |
| PEG 3350 | 6.5 | | 0 | |
| PEG 8000 | 0 | | 10 | |
| Polysorbate 80 | 1 | | 1 | |
| BHT antioxidant | 0.01 | | 0.01 | |
| EDTA dihydrate | 0.011 | | 0.011 | |
| PHMB (preservative) | 5 | ppm | 5 | ppm |
| pH | 7.1 | | 7.1 | |
| Osmolality (mOsm/kg) | about 300 | | about 300 | |
| Compound having Formula V | 0.001-1 | | 0.001-1 | |
| Water | q.s. to 100 | g | q.s. to 100 | g |

Note:
all quantities of ingredients are in grams, except PHMB

### EXAMPLE 8

Another Composition With Increased Viscosity That
May Be Used as Drug Delivery Vehicle

| Ingredient | Weight (g) | |
|---|---|---|
| PEG 3350 | 10 | |
| Polysorbate 80 | 1 | |
| BHT antioxidant | 0.01 | |
| Potassium chloride | 0.04 | |
| Sodium chloride | 0.1 | |
| Benzalkonium chloride (preservative) | 0.02 | |
| Na₂HPO₄ (anhydrous) | 0.182 | |
| NaH₂PO₄ (anhydrous) | 0.087 | |
| Alginate LF200S | 0.25 | |
| Polyvinyl pyrrolidone K90 | 0.75 | |
| Water | q.s. to 100 | g |

An exemplary composition of the present invention comprising the fluoroquinolone having Formula II is shown below.

### EXAMPLE 9

A Suspension Comprising Compound Having Formula II

| Ingredient | Weight (g) | |
|---|---|---|
| Poloxamer 188 (Pluronic ® F68) | 10 | |
| Poloxamer 407 (Pluronic ® F127) | 10 | |
| PEG-3350 | 5 | |
| PEG-40 stearate (Mryi ® 52) | 1 | |
| HPMC 15LV | 1 | |
| Sodium chloride | 0.1 | |
| Boric acid | 0.5 | |
| EDTA disodium dihydrate | 0.01 | |
| BHT | 0.01 | |
| PHMB HCl (preservative) | 0.0005 | |
| Compound having Formula II | 0.5 | |
| Water | q.s. to 100 | g |

Exemplary compositions of the present invention suitable for the treatment of elevated IOP are shown below or for ocular neuroprotection.

### EXAMPLE 10

A Composition Comprising Brimonidine Tartrate

| Ingredient | Weight (g) | |
|---|---|---|
| Polyglycerin 750 (decaglycerol) | 7.5 | |
| PEG-35 castor oil (Cremophor ELP) | 1 | |
| EDTA disodium dihydrate | 0.01 | |
| Benzalkonium chloride | 0.02 | |
| Tris(tromethamine) | 0.15 | |
| 1N NaOH or 1N HCl | for adjusting pH to 7.5 | |
| Brimonidine Tartrate | 0.5 | |
| Water | q.s. to 100 | g |

### EXAMPLE 11

A Composition Comprising Timolol Maleate

| Ingredient | Weight (g) | |
|---|---|---|
| PEG-8000 | 10 | |
| Polyglycerin 750 (decaglycerol) | 3 | |
| PEG-40 stearate (Mryj ® 52) | 1 | |
| EDTA disodium dihydrate | 0.01 | |
| BHT antioxidant | 0.01 | |
| Boric acid | 0.5 | |
| PHMB HCl (preservative) | 0.0001 | |
| Timolol maleate | 0.5 | |
| 1N NaOH | for adjusting pH to 7 | |
| Water | q.s. to 100 | g |

Exemplary compositions of the present invention suitable for the treatment of other ophthalmic disorders are shown below.

### EXAMPLE 12

A Suspension Comprising Levocabastine for
Allergic Conjunctivitis

| Ingredient | Weight (g) | |
|---|---|---|
| Polypropylene glycol (Polyglycol P425) | 5 | |
| PEG-35 castor oil (Cremophor ELP) | 1 | |
| HPMC 15 LV | 1 | |
| EDTA disodium dihydrate | 0.05 | |
| Benzalkonium chloride | 0.01 | |
| Boric acid | 0.5 | |
| Levocabastine | 0.05 | |
| 1N NaOH | for adjusting pH to 7 | |
| Water | q.s. to 100 | g |

### EXAMPLE 13

A Composition Comprising Loteprednol Etabonate and
Tobramycin for Ocular Inflammation and Infection

| Ingredient | Weight (g) | |
|---|---|---|
| PEG-8000 | 10 | |
| Polysorbate 80 | 1 | |
| Propylene glycol | 0.25 | |
| Glycerin | 0.25 | |
| EDTA disodium dihydrate | 0.01 | |
| BHT antioxidant | 0.01 | |
| Boric acid | 0.5 | |
| PHMB HCl (preservative) | 0.0001 | |
| Loteprednol Etabonate | 0.5 | |
| Tobramycin | 0.3 | |
| 1N HCl | for adjusting pH to 7 | |
| Water | q.s. to 100 | g |

Moreover, a composition, as described in Example 4, 6, 7, or 8 may be used to treat the dry eye condition by instilling one or more drops, one or more times daily to the anterior ocular surface of a patient suffering from dry eye to relieve the discomfort resulting from such condition.

The invention has now been described in such full, clear, concise and exact terms as to enable any person skilled in the art to which it pertains, to practice the same. It is to be understood that the foregoing describes examples of the invention and that modifications or substitutions may be made therein without departing from the spirit or scope of the invention as set forth in the claims. Moreover, while particular elements, embodiments and applications of the present invention have been shown and described, it will be understood, of course, that the present invention is not limited thereto since modifications or substitutions can be made by those skilled in the art without departing from the scope of the present disclosure, particularly in light of the foregoing teachings and appended claims. Moreover, it is also understood that the embodiments as described above are merely for illustrative purposes and not intended to limit the scope of the invention, which is defined by the following claims as interpreted according to the principles of patent law, including the Doctrine of Equivalents. Further, all references cited herein are incorporated in their entirety.

What is claimed is:

1. A composition comprising:
a) a water-soluble nonionic oxygen-containing polymer in a concentration from about 3 to about 25 percent by weight of the composition;
b) a surfactant in a concentration from about 0.01 to about 5 percent by weight of the composition;
c) a tonicity-adjusting agent; and
d) water;
wherein the composition has an osmolality in a range from about 200 to about 400 mOsm/kg.

2. The composition of claim 1, further comprising a medicament has low solubility in water, wherein the medicament is present in the composition in an amount sufficient to deliver a therapeutically effective amount of the medicament when the composition is administered into a target tissue.

3. The composition of claim 1, wherein the nonionic oxygen-containing polymer is selected from the group consisting of polyethylene glycol, polypropylene glycol, polyoxyethylene-polyoxypropylene block copolymers, and mixtures thereof.

4. The composition of claim 1, wherein the surfactant is selected from the group consisting of polysorbates, poloxamines, carboxylates, alkyl sulfonates, alkylaryl sulfonates, alkyl sulfates, quaternary ammonium salts, phospholipids, medium-chain triglycerides, and long-chain triglycerides.

5. The composition of claim 2, wherein the medicament is selected from the group consisting of anti-inflammatory agents, anti-infective agents, anti-allergic agents, antiproliferative agents, anti-angiogenic agents, antiglaucoma agents, anti-oxidants, antihypertensive agents, neuroprotective agents, cell receptor agonists, cell receptor antagonists, immunomodulating agents, immunosuppressive agents, IOP lowering agents, beta adrenoceptor antagonists, alpha-2 adrenoceptor agonists, carbonic anhydrase inhibitors, cholinergic agonists, prostaglandins and prostaglandin receptor agonists, angiotensin converting enzyme (“ACE”) inhibitors, AMPA receptor antagonists, NMDA antagonists, angiotensin receptor antagonists, somatostatin agonists, mast cell stabilizers, alpha-adrenergic receptor blockers, alpha-2 adrenoceptor antagonists, thromboxane A2 mimetics, protein kinase inhibitors, prostaglandin F derivatives, prostaglandin-2 alpha antagonists, cyclooxygenase-2 inhibitors, muscarinic agents, and combinations thereof.

6. The composition of claim 3, wherein the surfactant is selected from the group consisting of polysorbates, poloxamines, carboxylates, alkyl sulfonates, alkylaryl sulfonates, alkyl sulfates, quaternary ammonium salts, phospholipids, medium-chain triglycerides, and long-chain triglycerides.

7. The composition of claim 6, further comprising a medicament having Formula II.

8. The composition of claim 6, further comprising a medicament having Formula V.

9. A composition comprising:
a) a water-soluble nonionic oxygen-containing polymer in a concentration from about 3 to about 25 percent by weight of the composition;
b) a surfactant in a concentration from about 0.01 to about 5 percent by weight of the composition;
c) a tonicity-adjusting agent;
d) water; and
e) a medicament that has low solubility in water, said medicament being present at a concentration from 0.01 mg/g to about 100 mg/g of the composition; wherein the composition has an osmolality in a range from about 200 to about 400 mOsm/kg; the nonionic oxygen-containing polymer is selected from the group consisting of polyethylene glycol, polypropylene glycol, polyoxyethylene-polyoxypropylene block copolymers, and mixtures thereof; and the surfactant is selected from the group consisting of polysorbates, poloxamines, carboxylates, alkyl sulfonates, alkylaryl sulfonates, alkyl sulfates, quaternary ammonium salts, phospholipids, medium-chain triglycerides, and long-chain triglycerides.

10. The composition of claim 9, wherein the medicament comprises a compound having Formula II or V.

11. The composition of claim 9, wherein the medicament comprises brimonidine.

12. The composition of claim 9, wherein the medicament comprises loteprednol etabonate.

13. The composition of claim 9, wherein the medicament comprises moxifloxacin.

14. The composition of claim 9, wherein the medicament comprises gatifloxacin.

15. A method for treating an ocular disease, condition, or disorder in a patient, the method comprising administering into an ocular environment of said patient a composition that comprises:
a) a water-soluble nonionic oxygen-containing polymer in a concentration from about 3 to about 25 percent by weight of the composition;
b) a surfactant in a concentration from about 0.01 to about 5 percent by weight of the composition;
c) a tonicity-adjusting agent;
d) water; and
e) an ophthalmic medicament at a concentration from about 0.01 mg/g to about 100 mg/g;
wherein the composition has an osmolality in a range from about 200 to about 400 mOsm/kg; the nonionic oxygen-containing polymer is selected from the group consisting of polyethylene glycol, polypropylene glycol, polyoxyethylene-polyoxypropylene block copolymers, and mixtures thereof; and the surfactant is selected from the group consisting of polysorbates, poloxamines, carboxylates, alkyl sulfonates, alkylaryl sulfonates, alkyl sulfates, quaternary ammonium salts, phospholipids, medium-chain triglycerides, and long-chain triglycerides.

16. The method of claim 15, wherein the medicament is a member selected from the group consisting of: anti-inflammatory agents, anti-infective agents, anti-allergic agents, antiproliferative agents, anti-angiogenic agents, antiglaucoma agents, anti-oxidants, antihypertensive agents, neuroprotective agents, cell receptor agonists, cell receptor antagonists, immunomodulating agents, immunosuppressive agents, IOP lowering agents, beta adrenoceptor antagonists, alpha-2 adrenoceptor agonists, carbonic anhydrase inhibitors, cholinergic agonists, prostaglandins and prostaglandin receptor agonists, angiotensin converting enzyme (“ACE”) inhibitors, AMPA receptor antagonists, NMDA antagonists, angiotensin receptor antagonists, somatostatin agonists, mast cell degranulation inhibitors, alpha-adrenergic receptor blockers, alpha-2 adrenoceptor antagonists, thromboxane A2 mimetics, protein kinase inhibitors, prostaglandin F derivatives, prostaglandin-2 alpha antagonists, cyclooxygenase-2 inhibitors, muscarinic agents, and combinations thereof.

17. The method of claim 15, wherein the medicament comprises a compound having Formula V and the disease, condition, or disorder is inflammation of an eye.

18. The method of claim 15, wherein the medicament comprises a compound having Formula II and the disease, condition, or disorder is infection of an eye.

19. The method of claim 15, wherein the medicament comprises a quinolone or an analog thereof and the disease, condition, or disorder is infection of an eye.

20. A method for relieving ocular discomfort due to a dry eye condition in a patient, the method comprising administering into an ocular environment of said patient a composition that comprises:
a) a water-soluble nonionic oxygen-containing polymer in a concentration from about 3 to about 25 percent by weight of the composition;
b) a surfactant in a concentration from about 0.01 to about 5 percent by weight of the composition;
c) a tonicity-adjusting agent; and
d) water;
wherein the composition has an osmolality in a range from about 200 to about 400 mOsm/kg; the nonionic oxygen-containing polymer is selected from the group consisting of polyethylene glycol, polypropylene glycol, polyoxyethylene-polyoxypropylene block copolymers, and mixtures thereof; and the surfactant is selected from the group consisting of polysorbates, poloxamines, carboxylates, alkyl sulfonates, alkylaryl sulfonates, alkyl sulfates, quaternary ammonium salts, phospholipids, medium-chain triglycerides, and long-chain triglycerides.
